(19) United States
(12) Patent Application Publication
(10) Pub. No.: US 2005/0058609 A1
(43) Pub. Date: Mar. 17, 2005

(54) Medicated toothpick

(76) Inventor: Alireza Nazeri, St. Claire Shores, MI (US)

Correspondence Address: DYKAS, SHAVER & NIPPER, LLP, P.O. BOX 877, 802 WEST BANNOCK STREET, SUITE 405, BOISE, ID, 83701, US

(21) Appl. No.: 10/898,665

(22) Filed: Jul. 21, 2004

Related U.S. Application Data
(60) Provisional application No. 60/489,384, filed on Jul. 22, 2003.

(51) Int. Cl. A61K 7/16

(52) U.S. Cl. 424/49

(57) ABSTRACT

A medicated toothpick for administering a selected dosage of medication including a toothpick shaped device impregnated with a selected quantity of medication. The medicated toothpick is particularly useful for the delivery of nicotine and provides a physical and mental nicotine delivery system that may effectively be substituted for smoking.

### PRIORITY

This application claims the priority date of the provisional application entitled Medicated Toothpick filed by Alireza Nazeri on Jul. 22, 2003 with application Ser. No. 60/489,384.

### DESCRIPTION

### BACKGROUND OF THE INVENTION

1. Field of the Invention

The present invention generally relates to medication delivery systems, and more specifically to nicotine delivery systems, and particularly to a nicotine toothpick delivery system that provides for an improved nicotine release profile over existing systems.

2. Background Information

Delivery systems containing medications for oral administration include various chewing gum formulations and other similar methods and elements. Chewing gums permit release of the active ingredient over time as the gum product is masticated or chewed, and release is facilitated by the action of the saliva on the gum.

A problem with many chewing gum formulations is that they fail to deliver an adequate dose of medicament or active ingredient in the appropriate manner over the entire dosing interval. This results in the active ingredient insufficiently being absorbed into the bloodstream for effective therapeutic or pharmacological actions. There are many reasons for inadequate dosing. Many chewing gum formulations release the active medication slowly over time in a more or less continuous fashion; These formulations may also retain a significant portion of the active ingredient during the prescribed dosing period, resulting in inadequate dosing of the patient. Further, the particular gum base material chosen to contain and subsequently release the active material may not perform optimally. The gum base may be difficult to chew or unusually hard, thereby damaging the teeth and gums or making it unpleasant to chew. Additionally, some patients are just not in the habit of chewing gum and do not enjoy it.

As a result of the foregoing problems, many medication delivery systems for active substances are ineffective when delivered by chewing gum. This is unfortunate as many active ingredients would be quite amenable to an oral delivery system, especially those that enter the body through the mucous membranes lining the oral cavity, thereby avoiding first-pass metabolism that occurs with many oral formulations. Nicotine is one such example.

Nicotine is a highly addictive chemical stimulant present in cigarettes. Most smokers find quitting to be difficult, and attempts to quit often fail. The emergence of a craving for nicotine and of nicotine withdrawal symptoms makes sustained cessation difficult. Providing nicotine by medication is a proven method of smoking cessation, but one with limited success. Nicotine replacement is considered to work by relieving cravings and symptoms of withdrawal. Nicotine medications can affect cravings in various ways. For example, by providing a relatively steady level of nicotine in the bloodstream, such medications can prevent or blunt cravings throughout the day. For this purpose, a medication that provides steady sustained release and that maintains blood levels is most desirable. Smokers may also be subject to episodic peaks or surges of cravings, typically evoked by internal or external stimuli. Research has shown that these episodes often lead to relapse. Rapid relief of cravings in such episodes is expected to help prevent relapse. Acute delivery of nicotine via the oral mucosa may help relieve cravings, with the speed of relief being a function of the speed of delivery of nicotine into the blood stream.

Additionally, smoking is a physical and mental addiction. Many smokers are used to being able to “play” with the cigarette as they smoke it and are used to the physical contact as the nicotine is absorbed into their bloodstream. Most nicotine delivery systems, such as nicotine gum, do not provide that physical interaction. There is consequently a need in the art for an improved medication delivery system for actives such as nicotine that is both mentally and physically acceptable for a user that is to ingest the medication.

Additional objects, advantages and novel features of the invention will be set forth in part in the description which follows and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

The present invention provides a medicated toothpick for administering a selected medication to a user including a toothpick shaped device impregnated with a selected quantity of medication. The medication is soluble in saliva for efficient delivery of the medication to the user's system. The medicated toothpick is particularly suitable to helping a smoker quit smoking when a toothpick impregnated with nicotine is used as a substitute for smoking. Each medicated toothpick may be marked indicating the level of nicotine it contains so that a user can begin with a medicated toothpick with a higher dosage and work down to a small dosage or a standard non-medicated or mint flavored toothpick. In another embodiment, the medication is a nicotine imitation or substitute allowing a smoker to effectively eliminate the nicotine cravings. Additionally, the medication may be nitroglycereine, aspirin, diabetes medication, stimulants, relaxants, painkillers, herbal medications or any other medication that a user needs to effectively ingest.

In another embodiment, the present invention provides a pack of medicated toothpicks, including a pack for carrying the medicated toothpicks and a number of toothpick shaped devices. Each of the toothpick equivalents is impregnated with a quantity of nicotine to achieve a selected dosage when placed in a user's mouth. The time necessary to ingest the nicotine corresponds substantially with the time required to obtain such a dosage by smoking. The number of toothpicks is approximately equal to the number of cigarettes in a pack of cigarettes, in order to more completely replace the habit of handling and smoking a cigarette with the habit of handling and placing the medicated toothpick in the smoker's mouth.

The medicated toothpick may further include an internal compartment that may be broken for an immediate release of the medication. In additional embodiments the medicated toothpick is configured for prolonged release of the medication even after the immediate release of medication from the internal compartment. The medicated toothpick may concentrate the selected medication in one end of the toothpick so that the remaining portion of the toothpick is outside of the mouth. The medicated toothpick may include an additive such as a flavor, a breath freshener, a mint or citrus flavor, to mask the taste of the medication and enhance the experience, or for personal hygiene.

The purpose of the foregoing Abstract is to enable the United States Patent and Trademark Office and the public generally, and especially the scientists, engineers, and practitioners in the art who are not familiar with patent or legal terms or phraseology, to determine quickly from a cursory inspection, the nature and essence of the technical disclosure of the application. The Abstract is neither intended to define the invention of the application, which is measured by the claims, nor is it intended to be limiting as to the scope of the invention in any way.

Still other objects and advantages of the present invention will become readily apparent to those skilled in this art from the following detailed description wherein I have shown and described only the preferred embodiment of the invention, simply by way of illustration of the best mode contemplated by carrying out my invention. As will be realized, the invention is capable of modification in various obvious respects all without departing from the invention. Accordingly, the drawings and description of the preferred embodiment are to be regarded as illustrative in nature, and not as restrictive in nature.

### DESCRIPTION OF THE PREFERRED EMBODIMENTS

While the invention is susceptible to various modifications and alternative constructions, certain illustrated embodiments will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

The present invention is a medicated toothpick that is designed for administering a selected medication. In one embodiment, the present invention helps a smoker quit smoking. The device is a generally toothpick shaped device that is impregnated with a selected quantity of a nicotine replacing medication. The medication can be nicotine, which would replace the nicotine obtained from smoking cigarettes, chewing tobacco or any other tobacco products. It can also be other medications that replace, imitate or simulate nicotine or are approved and/or will be approved to use in nicotine withdrawal in usage for quitting smoking, such as buproprion or herbal medications. In another embodiment, medically useful medications may be delivered via the medicated toothpick, such as nitroglycerine, aspirin, diabetes medication or other medication. A feature of the device is that the selected medication is soluble in the user's saliva for easy ingestion into the user's system.

A toothpick like device is useful because it is something that is instinctively placed in a user's mouth, similar to cigarettes for smokers. Additionally, it is a physical object that allows the user to concentrate on something besides his/her craving. Many people already have the habit of having a toothpick or pencil in their mouth, making the transition from smoking to using a medicated toothpick very easy. In some cases, a person will have an aversion to other manners of ingesting drugs. For example, the gag reflex of many individuals prevents them from effectively swallowing medication.

A medicated toothpick allows a user to place it within their mouth at which point the toothpick can merely be placed there until their saliva mixes with the medication and is absorbed. Additionally, they may suck on the toothpick or chew it in order to release the medication. A smoker that is used to the physical interaction may “play” with the toothpick as it is absorbed, effectively replacing the physical interaction that they have come to associate with nicotine ingestion. Other toothpick like ways of delivering nicotine through the mouth (orally) can include such items as coffee straws, stir sticks, lollypop sticks, and other non-edible items frequently placed within the mouth. In addition to the selected medication such as nicotine, the toothpick can include other additives such as breath freshener; mint, citrus, clove or cinnamon flavor; or other desirable flavors to mask the taste of the medication, enhance the experience or for personal hygiene purposes. As a result of these novel features, the medicated toothpick is an effective way to encourage the ingestion of a medicated substance or nicotine substitute as defined above.

An optional feature of the medicated toothpick is that it can have one or more internal compartments that can be broken by the user to provide an immediate release of medication. With the internal compartment(s) broken, the toothpick can also be designed to provide an ongoing release of medication.

One desirable embodiment of the toothpick is one in which the medication is concentrated in one end of the toothpick, the tip. This allows a user to keep most of the toothpick out of his/her mouth, while getting the full release of medication from the tip that is in the user's mouth.

Another embodiment of the invention includes a smoking addiction breaking system, which includes use of the medicated toothpick described above. The addiction breaking system would include toothpicks of different levels of dosages of nicotine. The user could select the dosage and gradually move to a lower dosage in order to eliminate the nicotine intake from the smoking habit. In this system, the different dosages can be marked for recognition. The marking can be by the use of color-coding, or other dosage indicia, which indicates different dosages of medication.

One embodiment of the device is the use of the toothpicks described above in a pack. The pack would have certain analogies to a pack of cigarettes, and thus would be useful in helping the smoker substitute the habit of handling, manipulating and chewing a toothpick for the habit of handling, manipulating and smoking a cigarette. Such a pack of toothpicks would be equivalent to a pack of cigarettes in several ways. It would have the same number of toothpicks and the same amount of time would be required to obtain the full medication from the toothpick as would be required for a smoker to smoke a cigarette. Thus, when a smoker had the urge to have a cigarette, instead he/she could get out a pack of toothpicks, handle the toothpick by opening the pack and selecting one toothpick, and place it in his/her mouth. This satisfies the physical habit of handling something. This also satisfies the oral habit of chewing or sucking on an object. Instead of taking the cigarette in and out of the mouth as a smoker smokes, he/she could take the toothpick in and out of his/her mouth.

For instance, it has been determined that a typical smoker takes about 5 to 8 minutes to finish smoking a cigarette. Each cigarette contains roughly 8 to 9 mg of nicotine. Smoking each cigarette transfers about 1 to 1.5 mg of Nicotine to the smoker's body. Chewable tobacco transfers about 4.5 mg of nicotine. Dosages that transfer about these same levels of nicotine to the body would be the higher range of nicotine available, with lower dosages available to gradually reduce a person's dependency on smoking. A toothpick containing 1 mg or more each will be an approximate quantity to satisfy this need, and can be made available in different strengths.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

1. A medicated toothpick for administering a selected medication to a smoker, comprising;
a toothpick shaped device impregnated with a selected quantity of medication.

2. The medicated toothpick of claim 1 in which the selected medication is nicotine or a nicotine replacing medication.

3. The medicated toothpick of claim 1 in which the selected medication is any of nitroglycereine, aspirin, diabetes medication, stimulants, relaxants, pain killers or herbal medications.

4. The medicated toothpick of claim 1 in which said selected medication is soluble in saliva.

5. The medicated toothpick of claim 1, which is impregnated with a quantity of nicotine which when transferred to the user is approximately equal to the amount of nicotine transferred by using a tobacco product.

6. The medicated toothpick of claim 5 in which said medicated toothpick releases all of said quantity of nicotine in a time substantially equivalent to smoking an entire cigarette.

7. The medicated toothpick of claim 1 in which said selected medication is a nicotine substitute.

8. The medicated toothpick of claim 7, which is impregnated with a sufficient quantity of buproprion to impart a biochemical effect on a smoker.

9. The medicated toothpick of claim 1, which further comprises an internal compartment for containing said medications.

10. The medicated toothpick of claim 9 in which said internal compartment may be broken for an immediate release of medication.

11. The medicated toothpick of claim 10 in which said medicated toothpick is configured for prolonged release of said medication after said immediate release of medication from said internal compartment.

12. The medicated toothpick of claim 1 in which said selected medication is concentrated in one end of said toothpick.

13. The medicated toothpick of claim 1 which includes an additive such as a flavor, a breath freshener, a mint, citrus flavor, etc.

14. The medicated toothpick of claim 1, wherein said toothpick shaped device is any of a coffee straw, stir stick, non-edible chewing item, or lollypop stick.

15. A smoking addiction breaking system that includes a nicotine replacement medicated toothpick that comes in a plurality of dosage levels so a user can gradually use less nicotine replacement medication by selecting a dosage level that contains less medication.

16. The smoking addiction breaking system of claim 15 in which said different dosage levels are marked for recognition.

17. The smoking addiction breaking system of claim 16 in which said dosage levels are marked for recognition by the use of color.

18. A pack of medicated toothpicks, comprising:
a pack for carrying said medicated toothpicks;
a number of toothpick shaped devices in which each is impregnated with a quantity of nicotine to achieve a selected dosage when placed in a user's mouth for an amount of time substantially corresponding to a required time to receive said selected dosage from smoking cigarettes; wherein the number of toothpicks is approximately equal to the number of cigarettes in a pack of cigarettes, in order to more completely replace the habit of handling and smoking a cigarette with the habit of handling and placing said medicated toothpick in said user's mouth.

19. A medicated toothpick comprising:
a first end for placement in a user's mouth;
a second end for placement in a user's mouth; and
a toothpick body impregnated with a selected saliva soluble medication.

20. The medicated toothpick of claim 18, wherein any of said first end and said second end is pointed.

21. The medicated toothpick of claim 18, wherein said selected saliva soluble medication is nicotine for helping a smoker quit smoking by replacing his/her nicotine source.

22. The medicated toothpick of claim 18 in which said medication is concentrated in one end of said toothpick.
